(19) United States
(12) Patent Application Publication
(10) Pub. No.: US 2007/0010841 A1
(43) Pub. Date: Jan. 11, 2007

(54) Lancet assembly

(75) Inventors: Hock Meng Teo, Singapore (SG); Liak Hiong Goh, Singapore (SG)

Correspondence Address: LAWRENCE Y.D. HO & ASSOCIATES PTE LTD, 30 BIDEFORD ROAD, #07-01, THONGSIA BUILDING, SINGAPORE, 229922, SG

(73) Assignee: Medical Innovations Pte Ltd

(21) Appl. No.: 11/334,470

(22) Filed: Jan. 19, 2006

Related U.S. Application Data
(60) Provisional application No. 60/695,860, filed on Jul. 5, 2005.

(51) Int. Cl. A61B 17/32

(52) U.S. Cl. 606/181

(57) ABSTRACT

The present invention provides a lancet assembly comprising an internal housing, an external housing and a slider unit. The internal housing comprises an internal shaft configured to receive a sharp carrier of the slider unit. The sharp carrier comprises a shoulder that engages a lever arm of the shaft. When the external housing is moved towards the internal housing, an actuator pin in the external housing disengages the shoulder from the lever arm, thereby causing a spring to drive the sharp carrier towards the end of the internal housing. The sharp unit of the sharp carrier then extends through the aperture of the internal housing and pierces the body tissue. A flexible member, coupled to the shoulder, compresses when the sharp unit extends through the aperture. The flexible member thereafter relaxes and retracts the sharp unit back into the internal housing. Furthermore, a catch on the internal housing engages a depression defined in the external housing to prevent the reuse of the lancet assembly.

### FIELD OF THE INVENTION

The present invention generally relates to medical devices for use in taking blood samples by pricking a body tissue. In particular, the present invention is related to a lancet assembly for pricking a body tissue.

### BACKGROUND OF THE INVENTION

Lancets are typically utilized in the medical field for the purpose of drawing blood samples. The lancet usually comprises a needle that is fired upon a body tissue when triggered. A spring is commonly used to provide the necessary driving force for the needle to successfully pierce the body tissue.

Due to its applications in the medical field, lancet must be kept in sterilized conditions prior to being use. Furthermore, the lancet should include some safety features, such as retracting the needle back into the lancet to avoid accidental pricks on medical personnel, and preventing the reuse of the lancet to eliminate transmission of disease. One lancet device disclosed in U.S. Pat. No. 6,540,763 utilizes an elastic member to drive the sharps member, wherein the sharps member is used to pierce the body tissue. If the lancet device is not stored properly, the elastic member may harden and the lancet device may not work effectively.

Therefore, there is an imperative need to have a lancet assembly that is reliable and provides certain safety features. Other advantages of this invention will be apparent with reference to the detailed description.

### SUMMARY OF THE INVENTION

The present invention provides a lancet assembly comprising a sharp carrier, an internal housing, an external housing and a spring. The sharp carrier comprises a forward end, back end, at least one shoulder proximally coupled to the back end of the sharp carrier. An arm has one end coupled to the shoulder, and the other end of the arm consisting of an unattached flexible member. The forward end of the sharp carrier holds a sharp unit for piercing a body tissue. The at least one shoulder consists of a shoulder catch. The internal housing comprises an open end, an enclosed end and an internally located hollow shaft. The open end comprises a catch. The enclosed end comprises a centrally located aperture. The shaft extends from the enclosed end of the internal housing, wherein the shaft is configured to receive the sharp carrier. The shaft comprises at least one lever arm, wherein the lever arm comprises a peg for engaging the shoulder catch of the at least one shoulder. The external housing comprises an opening, a closed end and an internal frame, wherein the opening is configured to receive the open end of the internal housing. The closed end comprises a depression for receiving the catch of the internal housing. The frame extends from the closed end of the external housing. The frame comprises a cavity, and an actuator pin for disengaging the shoulder catch of the at least one shoulder from the peg of the lever arm. The spring is mounted between the sharp carrier back end and the cavity of the frame. To assemble the lancet assembly, the sharp carrier forward end is first inserted into the shaft at the open end of the internal housing. The open end of the internal housing is then covered by the external housing through the opening. The shoulder catch is initially engaged to the peg and the spring is compressed until such time as the external housing moves toward the internal housing, thereby causing the actuator pin to disengaged the shoulder catch from the peg. When the shoulder catch is disengaged from the peg, the lever arm rotates away from the at least one shoulder of the sharp carrier and causes stored energy in the spring to drive the sharp carrier towards the enclosed end of the internal housing. The sharp unit then extends through the aperture and pierces the body tissue, thereby compressing the flexible member. The flexible member thereafter relaxes to retract the sharp unit within the internal housing, wherein the catch of the internal housing engages the depression of the external housing to prevent the shoulder catch from re-engaging the peg.

Accordingly, in one aspect, the present invention provides an alternative embodiment of the lancet assembly, comprising a sharp carrier, an internal housing, an external housing and a spring. The sharp carrier comprises a forward end, back end, at least one shoulder proximally coupled to the back end of the sharp carrier. An arm has one end coupled to the shoulder, and the other end of the arm consisting of an unattached flexible member. The forward end of the sharp carrier holds a sharp unit for piercing a body tissue. The at least one shoulder consists of a shoulder catch. The internal housing comprises an open end, an enclosed end and an internally located hollow shaft. The open end comprises a catch. The enclosed end comprises a centrally located aperture. The shaft extends from the enclosed end of the internal housing, wherein the shaft is configured to receive the sharp carrier. The shaft comprises a first groove to receive the at least one shoulder, a frangible bar member to hold the at least one shoulder within the first groove of the shaft, and at least one guide slot configured to receive the shoulder of the sharp carrier. The external housing comprises an opening, a closed end and an internal frame. The opening is configured to receive the open end of the internal housing. The closed end comprises a depression for receiving the catch of the internal housing. The frame extends from the closed end of the external housing. The frame comprises a cavity and a second groove for pushing the shoulder towards the frangible bar member. The spring is mounted between the sharp carrier back end and the cavity of the frame. To assemble the lancet assembly, the sharp carrier forward end is inserted into the shaft at the open end of the internal housing. The open end of the internal housing is then covered by the external housing through the opening. The shoulder is initially rested upon the first groove and the spring is compressed until such time as the external housing moves toward the internal housing, thereby causing the second groove to force the shoulder to break the frangible bar member. The sharp carrier then rotates axially until the shoulder enters the guide slot, thereby causing stored energy in the spring to drive the sharp carrier towards the enclosed end of the internal housing. The sharp unit extends through the aperture and pierces the body tissue, thereby compressing the flexible member. The flexible member thereafter relaxes to retract the sharp unit within the internal housing, wherein the catch of the internal housing engages the depression of the external housing to prevent the shoulder catch from re-engaging the peg.

### BRIEF DESCRIPTION OF THE DRAWINGS

A preferred embodiment of the present invention will now be more fully described, with reference to the drawings of which:

FIG. 1 is a perspective view of the unassembled components of the lancet assembly of the present invention.

FIG. 2 is a cross-sectional view of the internal housing taken along line 2-2 in FIG. 1.

FIG. 3A is a cross-sectional view of the slider unit taken along line 3-3 in FIG. 1.

FIG. 3B is a particular enlarged cross-sectional view of the frangible connection between the sharp carrier and the protective cap.

FIG. 3C is a side view of the slider unit.

FIG. 3D is a front view of the slider unit.

FIG. 4 is a cross-sectional view of external housing taken along line 4-4 in FIG. 1

FIG. 5 is a bottom view of the internal housing.

FIG. 6A is a cross-sectional view of the lancet assembly in an armed position taken along the lines 2-2, 3-3 and 4-4.

FIG. 6B is an alternative cross-sectional view of the lancet assembly in an armed position taken along the lines 6-6 and 6′-6′.

FIG. 7A is a cross-sectional view of the lancet assembly in the final position taken along the lines 2-2, 3-3 and 4-4.

FIG. 7B is an alternative cross-sectional view of the lancet assembly in the final position taken along the lines 6-6 and 6′-6′.

FIG. 8A-C are fragmentary cross-sectional views of the trigger mechanism of the lancet assembly.

FIG. 9 is a cross-sectional view of the internal housing of an alternative embodiment.

FIG. 10 is a cross-sectional view of the external housing of an alternative embodiment

FIG. 11A-C are cross-sectional views of the trigger mechanism of the alternative embodiment.

### DETAILED DESCRIPTION OF THE INVENTION

The present invention may be understood more readily by reference to the following detailed description of certain embodiments of the invention.

1. Lancet Assembly

The present invention provides a lancet assembly that comprises an internal housing 20, a slider unit 40, a spring 50, and an external housing 60. FIG. 1 shows a preferred embodiment of the lancet assembly, wherein the line A-A represents the central longitudinal axis of the lancet assembly.

2. Internal Housing

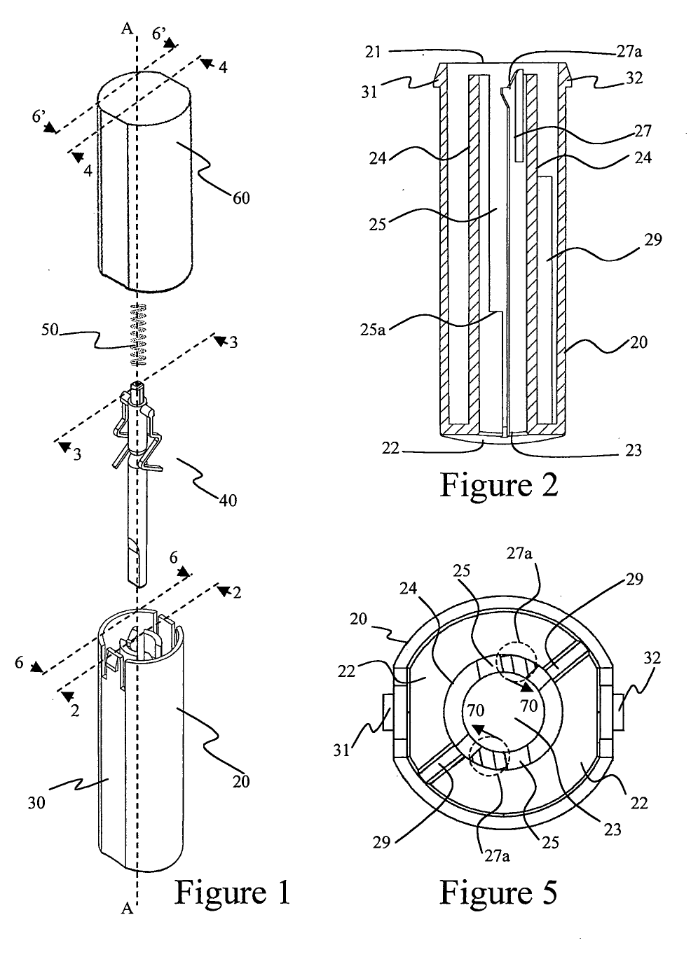

In the preferred embodiment, the internal housing 20 has a cylindrical shape consisting of two longitudinally aligned level planes 30. Referring to FIGS. 2 and 5, the internal housing 20 has an opening 21 at one end to receive the slider unit 40, and is enclosed in the other end 22. A first catch 31 and a second catch 32 are identically defined on each level plane 30 at the opening 21. The enclosed end 22 is the contact surface between the lancet assembly and the body tissue. An aperture 23 is centrally located at the enclosed end 22 to allow the sharp unit 43 of the slider unit 40 to pierce the body tissue, which will be described in detail hereinafter.

The internal housing 20 further comprises an internal hollow shaft 24 that is configured to receive the slider unit 40. The internal shaft 24 is cylindrically shaped and extends longitudinally from the aperture 23 of the enclosed end 22. A pair of pillars 29 are coupled between the shaft 24 and the internal housing 20 to provide additional support to the shaft. Opposed guide slots 25 are defined along the shaft 24 to allow translation of the slider unit 40 along the shaft, which will be discussed hereinafter. The guide slot 25 consists of an abutment 25a to control the depth of penetration of the sharp unit 43 into the body tissue. The shaft further comprises a lever arm 27 that comprises a peg 27a, wherein the peg is configured to support the slider unit 40 before the latch assembly is activated.

3. Slider Unit

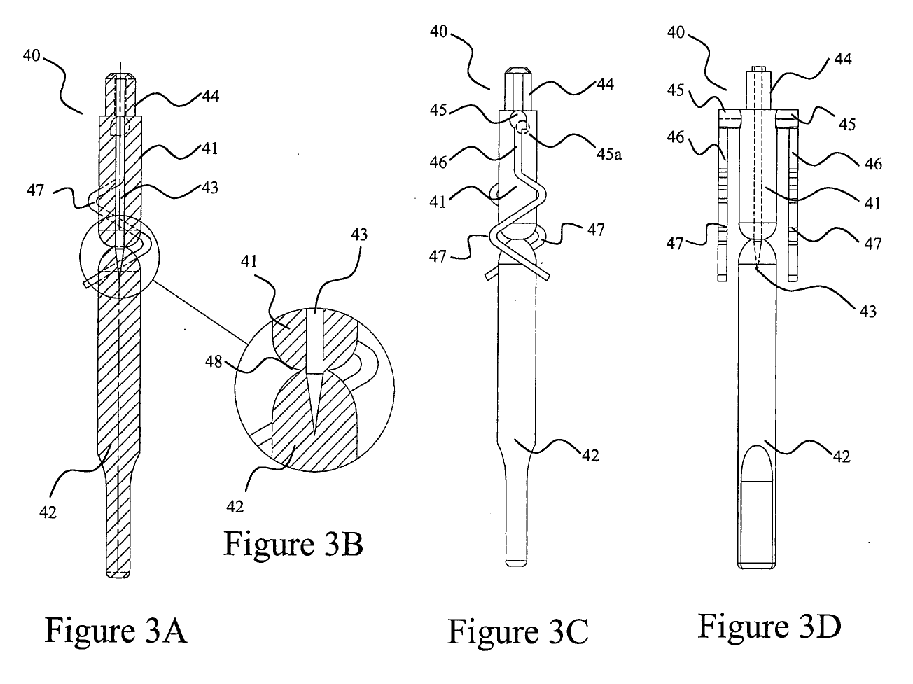

Referring to FIGS. 3A-D, the slider unit 40 comprises a sharps carrier 41 and a protective cap 42. The sharps carrier 41 holds the sharp unit 43, wherein the sharp unit is typically a pointed needle made of stainless steel and coated with medical grade lubrication. The slider unit 40 is preferably manufactured in a single plastic injection molding process. A spring shoulder 44 is mounted on top of the slide carrier 41 to support the spring 50, wherein the spring provides energy for the slider carrier to move towards the enclosed end 22 of the internal housing 20.

The slide unit 40 further comprises a pair of arms 46 coupled to the sharp carrier 41 via a pair of carrier shoulders 45. Referring to FIG. 3C, one end of the arm 46 is fixedly coupled to the carrier shoulder 45 while the other end is unattached. The unattached end of the arm consists of a flexible zigzag member 47 that acts as a spring, which will be discussed hereinafter. A shoulder catch 45a is defined on the carrier shoulder 45, and is part of the trigger mechanism used in the present invention. The entire slider unit 40 is preferably made from plastic.

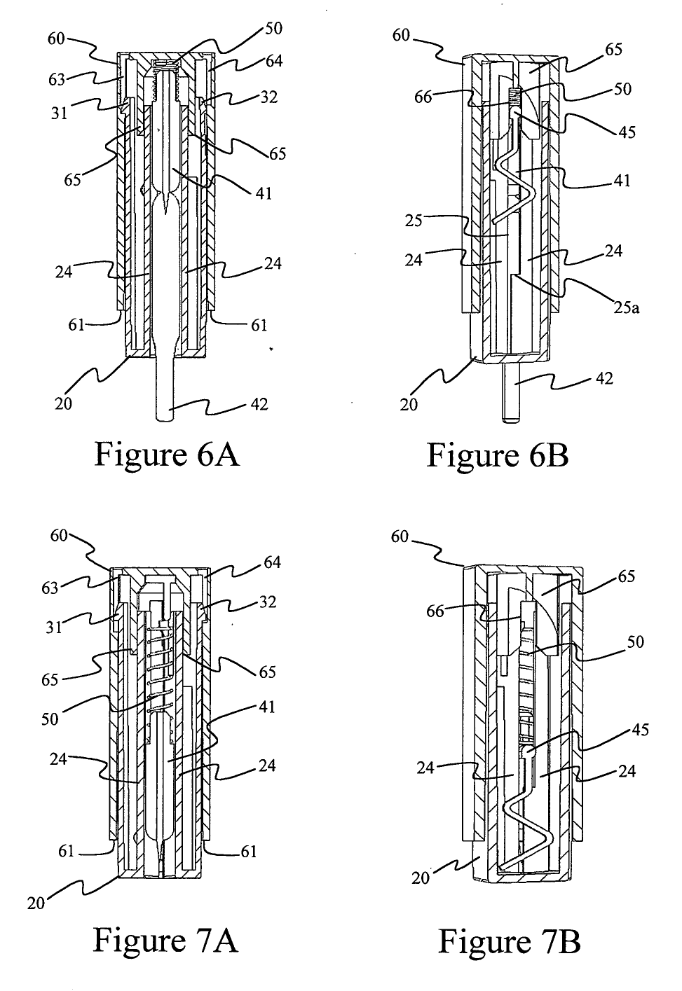

The protective cap 42 is attached to the sharp carrier 41 at a frangible junction 48 as illustrated in FIG. 3B. The protective cap 42 prevents the sharp unit 43 from contamination, and protects the sharp unit 43 during transportation of the lancet assembly. Briefly referring to FIG. 6A, the lancet assembly is in an armed position, wherein the length of the protective cap 42 extends through the aperture 23 of the internal housing 20. Before activating the lancet assembly, the protective cap 42 is twisted or the like to sever the frangible junction 48. In an alternative method, the protective cap 42 can be removed from the slide carrier 41 before assembling the lancet assembly.

4. External Housing

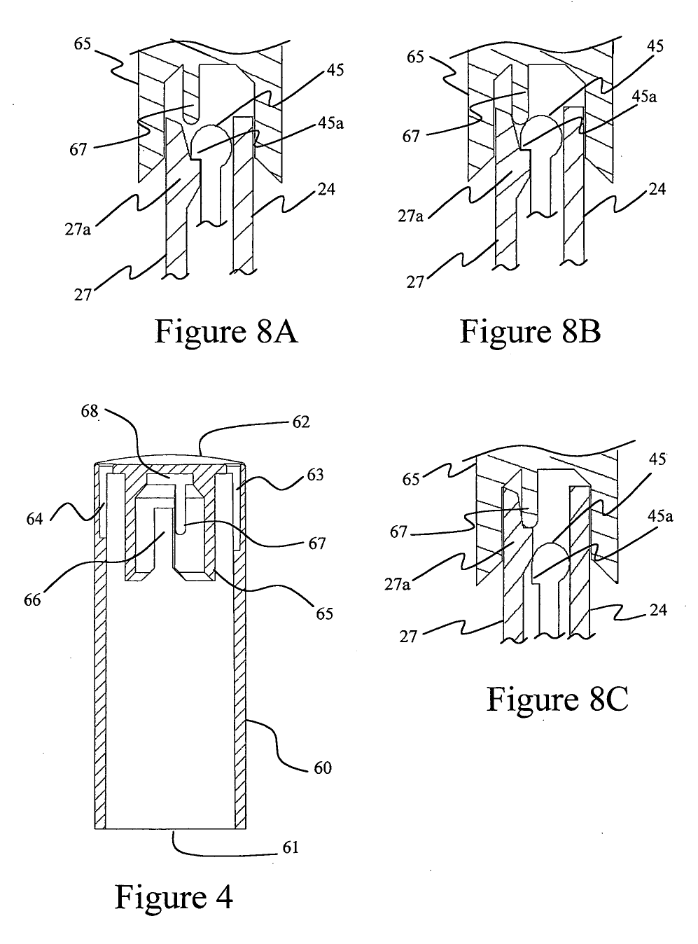

Referring to FIG. 6A, the external housing 60 has an opening 61 configured to receive the internal housing 20 and slider unit 40. FIG. 4 shows the external housing 60 having an enclosed end 62. A pair of depressions 63, 64 are defined at the walls of the external housing 60 near the enclosed end 62, wherein the pair of depressions are configured to receive the pair of catches 31, 32 of the internal housing 20. The depression 63 has a longer depth from the enclosed end 62 than the depression 64, which will be discussed hereinafter.

The external housing 60 comprises an internal frame 65 configured to receive the spring 50, the slide carrier 41 and the shaft 24 of the internal housing 20 (see FIG. 6A). The internal frame is cylindrical in shape and extends from the enclosed end 62 of the external housing 60. Opposed shoulder slots 66 are defined on the internal frame 65, wherein the shoulder slots are configured to receive the pair of carrier shoulders 45 of the sharp carrier 41. Referring to FIG. 6B, the shoulder slots 66 are aligned to the guide slots 25 to allow translation of the slide carrier 41. An actuator pin 67 is mounted within the internal frame 65 and acts as a trigger mechanism for the lancet assembly. A cavity 68 is defined at the enclosed end 62 of the internal housing 20, wherein the cavity is configured to secure the spring 50.

The preferred external housing 20 and internal housing 40 has a cylindrical configuration, but other shapes, for example square, rectangular, or oval, can also be used.

5. Operation

The operation of the lancet assembly will now be described with reference to FIGS. 6-7. FIG. 6A illustrates how the lancet assembly is assembled in an armed position before the actual use. FIG. 7A shows the final position of the lancet assembly after it is activated. The workings of the trigger mechanism is sequentially illustrated in FIGS. 8A-C.

6. Armed Position

Referring to FIGS. 6A and 6B, the slider unit 40 is inserted into the shaft 24 through the opening 21 of the internal housing 20. The slider unit 40, the spring 50 and the internal housing 20 are then inserted into the external housing 60 through the opening 61. To obtain an armed position, the first catch 31 of the internal housing 20 engages the longer depression 63 of the external housing 60. At the same time, the second catch 32 of the internal housing 20 is disengaged from the shorter depression 63 of the external housing 60.

FIG. 6A shows that the internal frame 65 of the external housing 60 is configured to receive the shaft 24 of the internal housing 20. FIG. 6B shows that the pair of shoulder slots 66 of the internal frame 65 is aligned to the pair of guide slots 25 in the shaft 24. Furthermore, the shoulder slots 24 are configure to receive the carrier shoulders 66 of the sharp carrier 41. Referring to FIG. 8A, the shoulder catch 45a of the sharp carrier 41 is engaged to the peg 27a of the lever arm 27 in the armed position.

One end of the spring 50 is mounted on the spring shoulder 44 of the sharp carrier 41 and the other end of the spring is supported by the cavity of the 68 of the external housing 60. In the armed position, the spring 50 is compressed and stores potential energy.

7. Activating Position

When the lancet assembly is ready for use, the protective cap 42 is first removed from the sharp carrier 41 as discussed above. The enclosed end 22 of the internal housing 20 is then placed in contact with the body tissue, wherein the internal housing maintains a stationary position. A finger is place on the enclosed end 62 of the external housing 60 to depress the external housing towards the internal housing 20.

Referring to FIG. 8B, the linear movement of the external housing 60 towards the stationary internal housing 20 results in a relative linear movement of the actuator pin 67 towards the carrier shoulder 45, wherein the carrier shoulder is engaged to the peg 27a of the lever arm 27.

When the external housing 60 is further depressed, the actuator pin 67 interacts with the carrier shoulder 45, wherein the actuator pin disengages the carrier shoulder from the peg 27a. Referring to FIG. 8C, the actuating pin 67 pushes the carrier shoulder 45 away from the peg 27a of the lever arm 27, resulting in the rotational movement 70 of the lever arm about the longitudinal axis A-A (see FIG. 5 also). The shoulder slot 66 prevents any rotational motion of the carrier shoulder 45.

After the shoulder catch 45a is released from the peg 27a, the sharp carrier 41 is able to freely translate through the shaft 24 of the internal housing 20. At the instantaneous moment that the shoulder catch 45a disengages from the peg 27a, the spring 50 relaxes, thereby releasing the stored energy in the spring. This stored energy transforms into kinetic energy for the sharp carrier 41, thereby allowing the sharp carrier 41 to translate linearly through the shaft 24, wherein the linear movement of the sharp carrier is parallel to the longitudinal axis A-A.

When the sharp carrier 41 reaches the enclosed end 22 of the internal housing 20, the sharp unit 43 extends through the aperture 23 and pierces the body tissue. The abutment 25a controls the penetration depth of the sharp unit 43 into the body tissue by limiting the movement of the carrier shoulder 45. At the same time, the flexible zigzag member 47 of the slide carrier 41 is compressed.

8. Final Position

The compressed zigzag member 47 of the slide carrier 41 thereafter relaxed and thereby pushes the sharp carrier 41 away from the enclosed end 22 of the internal housing 20, wherein the sharp unit 43 retracts into the aperture 23 (see FIG. 7B). In this final position, the second catch 32 of the internal housing 20 is engaged to the shorter depression 63 of the external housing 60. The engagement of the second catch 32 to the shorter depression 63 locks the internal housing 20 to the external housing 60, thereby preventing the lancet assembly from re-use.

9. Alternative Embodiment

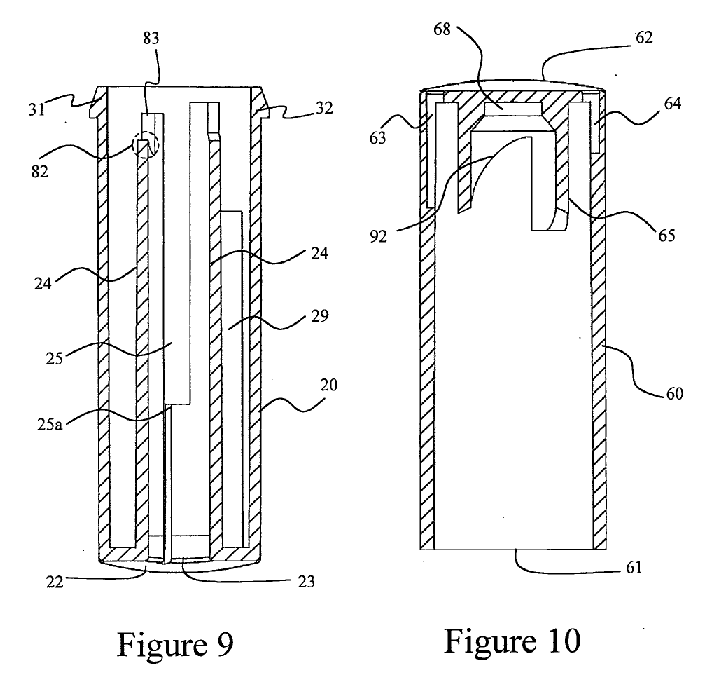

In an alternative embodiment, the shaft 24 of the internal housing 20 comprises a pair of opposite grooves 82, wherein the grooves 82 are configured to receive the carrier shoulders 45 of the sharp carrier 41 (see FIG. 9). Opposed bar members 83 are frangibly coupled to shaft 24, which will be discussed in detail hereinafter.

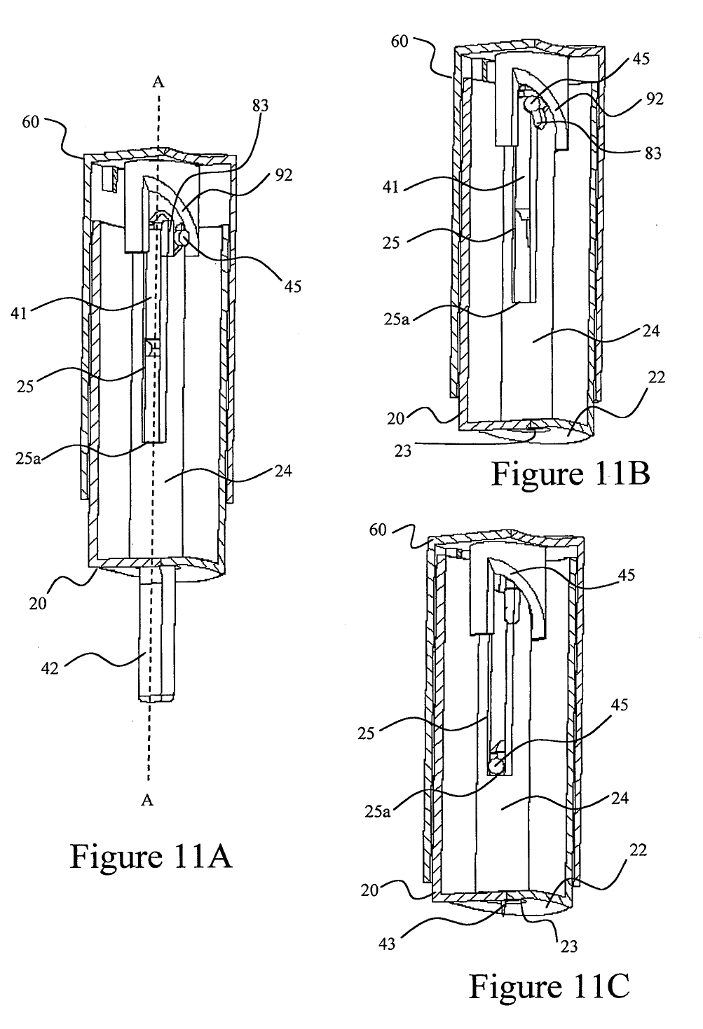

Referring to FIG. 10, the internal frame 65 of the external housing 60 comprises opposed V-shaped grooves 92. When the slider unit 40 is inserted into internal housing, the carrier shoulder 45 is retained within the groove 82 of the shaft 24. Thereafter, the internal housing 20 and slider unit 40 are inserted into the external housing 60, wherein the V-groove 92 is in contact with the carrier shoulder 45 as shown in FIG. 11A. For simplicity, the arm 46 and flexible zigzag member 47 of the sharp carrier 41 are not illustrated in FIGS. 11A-C. The first catch 31 of the internal housing 20 engages the longer depression 63 of the external housing 60 and the lancet assembly is in the armed position.

Before using the lancet assembly, the protective cap 42 is removed. The enclosed end 22 of the internal housing 20 is placed in contact with the body tissue, wherein the internal housing maintains a stationary position. A finger is placed on the enclosed end 62 of the external housing 60 to depress the external housing towards the internal housing 20.

Referring to FIG. 11B, the relative movement of the external housing 20 towards the internal housing 60 forces the carrier shoulder 45 to break the frangible bar member 83. Further depression of the external housing 60 axially rotates the carrier shoulder 45 until the carrier shoulder enters the guide slot 25. Thereafter, the spring 50 relaxes and provides energy for the sharp carrier 41 to translate through the shaft 24. When the sharp carrier 41 reaches the enclosed end 22 of the internal housing 20, the sharp unit 43 extends through the aperture 23 and pierces the body tissue. The abutment 25a controls the penetration depth of the sharp unit 43 into the body tissue by limiting the movement of the carrier shoulder 45 (see FIG. 11C).

When the sharp unit pierces the body tissue, the flexible zigzag member 47 of the slide carrier 41 is compressed. The zigzag member 47 thereafter relaxed and pushes the sharp carrier 41 away from the enclosed end 22 of the internal housing 20, wherein the sharp unit 43 retracts into the aperture 23. In this final position, the second catch 32 of the internal housing 20 is engaged to the shorter depression 63 of the external housing 60. The engagement of the second catch 32 to the shorter depression 63 locks the internal housing 20 to the external housing 60, thereby preventing the lancet assembly from re-use

While the present invention has been described with reference to particular embodiments, it will be understood that the embodiments are illustrative and that the invention scope is not so limited. Alternative embodiments of the present invention will become apparent to those having ordinary skill in the art to which the present invention pertains. Such alternate embodiments are considered to be encompassed within the spirit and scope of the present invention. Accordingly, the scope of the present invention is described by the appended claims and is supported by the foregoing description.

What is claimed is:

1. A lancet assembly comprising:
a sharp carrier comprising a forward end and back end, wherein the forward end of the sharp carrier holds a sharp unit for piercing a body tissue, the sharp carrier comprising:
at least one shoulder proximally coupled to the back end of the sharp carrier, wherein the at least one shoulder consists of a shoulder catch;
an arm with one end coupled to the shoulder, and the other end consisting of an unattached flexible member;
an internal housing comprising an open end, an enclosed end and an internally located hollow shaft, wherein the open end comprises a catch, wherein the enclosed end comprises a centrally located aperture, wherein the shaft extends from the enclosed end of the internal housing, wherein the shaft is configured to receive the sharp carrier, the shaft comprising:
at least one lever arm comprising a peg, wherein the peg engages the shoulder catch of the at least one shoulder;
an external housing comprising an opening, a closed end and an internal frame, wherein the opening is configured to receive the open end of the internal housing, wherein the closed end comprises a depression for receiving the catch of the internal housing, wherein the frame extends from the closed end of the external housing, the frame comprising:
a cavity;
an actuator pin for disengaging the shoulder catch of the at least one shoulder from the peg of the lever arm;
a spring mounted between the sharp carrier back end and the cavity of the frame; and
whereby the sharp carrier forward end is inserted into the shaft at the open end of the internal housing, thereby covered by the external housing through the opening, the shoulder catch is initially engaged to the peg and the spring is compressed until such time as the external housing moves toward the internal housing, thereby causing the actuator pin to disengaged the shoulder catch from the peg, wherein the lever arm rotates away from the at least one shoulder of the sharp carrier and causing stored energy in the spring to drive the sharp carrier towards the enclosed end of the internal housing, wherein the sharp unit extends through the aperture and pierces the body tissue, thereby compressing the flexible member, and the flexible member thereafter relaxes to retract the sharp unit within the internal housing, wherein the catch of the internal housing engages the depression of the external housing to prevent the shoulder catch from re-engaging the peg.

2. The lancet assembly of claim 1, wherein the shaft of the internal housing further comprises at least one guide slot configured to receive the shoulder of the sharp carrier, wherein the at least one guide slot comprises a abutment located near the enclosed end of the internal housing;
whereby the least one guide slots guides the movement of the sharp carrier towards the enclosed end of the internal housing; and
whereby the abutment controls the penetration depth of the sharp unit into the body tissue by limiting the movement of the shoulder of the sharp carrier as the sharp carrier moves towards the enclosed end of the internal housing.

3. The lancet assembly of claim 1, wherein the frame of the external housing further comprises at least one shoulder slot to receive the at least one shoulder of the sharp carrier, wherein the at least one shoulder slot prevents the at least one shoulder from twisting when the shoulder catch disengages from the peg.

4. The lancet assembly of claim 1 further comprising a removable cap coupled to the forward end of the sharp carrier, wherein the cap extends through the aperture of the enclosed end of the internal housing.

5. The lancet assembly of claim 1, wherein the sharp unit is a pointed needle made of stainless steel

6. The lancet assembly of claim 1, wherein the spring is metallic.

7. The lancet assembly of claim 4, wherein the sharp carrier and the removable cap are molded as one entity using plastic injection process.

8. The lancet assembly of claim 1, wherein the sharp unit is coated with medical grade lubrication.

9. The lancet assembly of claim 1, wherein the sharp carrier, the spring, and the internal housing are not coupled to each other.

10. A lancet assembly comprising:
a sharp carrier comprising a forward end and back end, wherein the forward end of the sharp carrier holds a sharp unit for piercing a body tissue, the sharp carrier comprising:
at least one shoulder proximally coupled to the back end of the sharp carrier;
an arm with one end coupled to the shoulder, and the other end consisting of an unattached flexible member;
an internal housing comprising an open end, an enclosed end and an internally located hollow shaft, wherein the open end comprises a catch, wherein the enclosed end comprises a centrally located aperture, wherein the shaft extends from the enclosed end of the internal housing, wherein the shaft is configured to receive the sharp carrier, the shaft comprising:
a first groove to receive the at least one shoulder;
a frangible bar member to hold the at least one shoulder within the first groove of the shaft;
at least one guide slot configured to receive the shoulder of the sharp carrier;
an external housing comprising an opening, a closed end and an internal frame, wherein the opening is configured to receive the open end of the internal housing, wherein the closed end comprises a depression for receiving the catch of the internal housing, wherein the frame extends from the closed end of the external housing, the frame comprising:
a cavity;
a second groove for pushing the shoulder towards the frangible bar member;
a spring mounted between the sharp carrier back end and the cavity of the frame; and
whereby the sharp carrier forward end is inserted into the shaft at the open end of the internal housing, thereby covered by the external housing through the opening, the shoulder is initially rested upon the first groove and the spring is compressed until such time as the external housing moves toward the internal housing, thereby causing the second groove to force the shoulder to break the frangible bar member, wherein the sharp carrier rotates axially until the shoulder enters the guide slot, thereby causing stored energy in the spring to drive the sharp carrier towards the enclosed end of the internal housing, wherein the sharp unit extends through the aperture and pierces the body tissue, thereby compressing the flexible member, and the flexible member thereafter relaxes to retract the sharp unit within the internal housing, wherein the catch of the internal housing engages the depression of the external housing to prevent the shoulder catch from re-engaging the peg.

11. The lancet assembly of claim 10, wherein the at least one guide slot comprises a abutment located near the enclosed end of the internal housing,
wherein the abutment controls the penetration depth of the sharp unit into the body tissue by limiting the movement of the shoulder of the sharp carrier as the sharp carrier moves towards the enclosed end of the internal housing.

12. The lancet assembly of claim 10 further comprising a removable cap coupled to the forward end of the sharp carrier, wherein the cap extends through the aperture of the enclosed end of the internal housing.

13. The lancet assembly of claim 10, wherein the sharp unit is a pointed needle made of stainless steel

14. The lancet assembly of claim 10, wherein the spring is metallic.

15. The lancet assembly of claim 12, wherein the sharp carrier and the removable cap are molded as one entity using plastic injection process.

16. The lancet assembly of claim 10, wherein the sharp unit is coated with medical grade lubrication.

17. The lancet assembly of claim 10, wherein the sharp carrier, the spring, and the internal housing are not coupled to each other.
